(19) United States
(12) Patent Application Publication
(10) Pub. No.: US 2025/0281737 A1
(43) Pub. Date: Sep. 11, 2025

(54) DEVICE AND METHOD OF TREATING PROLIFERATIVE DISORDERS OF THE PROSTATE GLAND

(71) Applicants: Neal A Scott, Mountan View, CA (US); Mark McElligott; Edward Karpman, Mountain View, CA (US)

(72) Inventors: Neal A Scott, Mountain View, CA (US); Mark McElligott, San Jose, CA (US); Edward Karpman, Los Altos, CA (US)

(21) Appl. No.: 18/600,678

(22) Filed: Mar. 9, 2024

(51) Int. Cl. A61N 1/05; A61F 2/04; A61F 2/958; A61M 25/00; A61N 1/36

(52) U.S. Cl. CPC A61N 1/0514; A61F 2/958; A61M 25/0017; A61N 1/36007; A61F 2002/047; A61M 2205/054

(57) ABSTRACT

One embodiment comprises a tubular, expandable metal mesh outside of a Foley catheter, which is held in place during treatment with a bladder balloon. The mesh is compressed for insertion into the prostate. The mesh is then expanded so that at least a portion of the mesh is against the prostatic urethral wall. The mesh is treated with a medicament, such as paclitaxel. Urine may flow through the catheter since there is no blockage of the urethra. Embodiments use different elements and methods for compressing or expanding the metal mesh. The mesh in a passive state may be either compressed or expanded. The state if the mesh is changed by either pushing or pulling on the mesh while it is in place in the urethra. Other embodiments may use the metal mesh as one electrode for iontophoresis or electroporation.

### BACKGROUND OF THE INVENTION

Benign prostatic hypertrophy (BPH) is a histological diagnosis defined by the unregulated proliferation of connective tissue, smooth muscle and glandular epithelium within the prostate transition zone (Fitzpatrick 2006) (Fukuta F 2012). Clinically, BPH is diagnosed when a bladder outlet obstruction (suspected by voiding symptoms or flow rate measurement) is attributed to prostatic enlargement, which compresses the prostatic urethra (FIG. 1).

Benign prostatic hypertrophy is one of the most common diseases of mankind. The exact prevalence varies by the definition used and the population studied. Berry et al. (Berry S J 1984) examined data from five prior studies and demonstrated that no men under age 30 had BPH, while the prevalence of BPH was 8 percent in the fourth decade. Fifty percent of men had evidence of pathologic BPH between ages 50 and 60. The prevalence of BPH rises markedly with increased age and men in the ninth decade of life have a prevalence of 80% (Berry S J 1984).

More serious complications of BPH can include acute urinary retention, defined as the sudden inability to urinate that requires catheterization; recurrent urinary tract infections; gross hematuria; and bladder stones (Committee 2003); (Hargreave T B 2005). The initial treatment of BPH primarily consists of medications (Connolly S S 2007). Surgical techniques are usually reserved for those patients who continue to experience symptoms that are refractory to medical therapy.

Transurethral resection of the prostate is the most commonly performed surgical procedure for BPH. However, this procedure is associated with a number of complications including hemorrhage requiring transfusion, requirement for a repeat procedure within one year, long term failure to void requiring chronic or intermittent catheterization, stress incontinence, bladder neck stenosis, storage lower urinary tract symptoms, erectile dysfunction, and urethral strictures (Young M J 2018).

As a result of the significant incidence of complications associated with the surgical techniques in use to reduce prostate size, minimally invasive therapies have been developed that aim to achieve similar efficacy with reduced side effects. These therapies include permanent implants that lift and hold open the prostatic urethra (e.g., prostatic urethral lift) and devices that use various forms of energy to resect or ablate tissue to relieve the bladder obstruction (Foster H E 2019). There is a continued clinical need to fill the gap between medication and surgical therapies for treatments that provide significant and durable symptom relief, while minimizing morbidity and improving the quality of life of the patient.

### Current Treatments for Benign Prostatic Hyperplasia

Transurethral resection of the prostate (TURP) and suprapubic enucleation procedures have been established as the gold standard in clinical practice. While TURP is mostly used for smaller and medium-sized prostate volumes (up to 80 mL), large adenomas are enucleated by open surgery. However, the latter procedure (“adenoma enucleation”) is now less frequently used because transurethral enucleation techniques (endoscopic enucleation of the prostate) are now becoming increasingly widespread (Miernik and Gratzke 2020). These procedures are associated with relatively high transfusion (9.5%) and revision (9.8%) rates for TURP for gland sizes greater than 60 g, and also relatively high transfusion rates (7.5%) and prolonged hospital stay (11.9 days) after adenoma enucleation (Gratzke C 2007), (Reich O 2008). Transurethral enucleation procedures such as holmium laser enucleation of the prostate, thulium laser enucleation of the prostate, or bipolar enucleation of the prostate have a better safety profile in this respect. Zhang et al. reviewed a total of 27 randomized controlled trials comparing endoscopic enucleation of the prostate with its subgroups versus TURP. Reviewing the evidence in a meta-analysis, it appears that technically correct performance of endoscopic enucleation of the prostate does not depend on the type of energy used (holium, thulium, bipolar current) (Zhang Y 2019).

### Catheter-Based Treatments for BPH

Initial catheter-based therapies consisted of dilatation of the prostatic urethra with angioplasty balloons (Reddy, Mikulich and Clark 1990), (Condon, Bley and Purkait 1993) or the implantation of bare metal stents (Rosenbluth, Method and apparatus for treating hypertropy of the prostate gland 1988), (Rosenbluth, Method and apparatus for treating hypertrohy of the prostate gland 1990). These techniques have not been shown to be useful for long-term use in humans. Catheter-based techniques utilizing heat have also been described (Fenn and Mon 2022), U.S. Pat. No. 5,007,437), (Bolmsjo and Schelin 2003), (Swoyer and Skwarek 2007), (Sterzer 1991), (Aljuri, Mantri, et al. 2017).

The Prostatic Urethral Lift procedure is conducted by installing small permanent implants transurethrally under endoscopic guidance to lift apart the obstructing lateral lobes and thereby reduce urethral obstruction (Catanese, et al. 2010). Roehrborn et al. performed a prospective, randomized controlled study of the Prostatic Urethral Lift in men with symptomatic lower urinary tract symptoms due to BPH. After 5 years of follow up, improvements in International Prostate Symptom Score, quality of life, BPH Impact Index, and maximal urinary flow rate were 36%, 50%, 52% and 44%, respectively. However, the need for surgical retreatment was 13.6% over 5 years. (Roehrborn C 2017).

Prostatic artery embolization is performed with vascular access through the femoral or radial arteries and small embolization particles are injected directly into the prostatic arteries bilaterally in order to devascularize adenomatous tissue (Reb, et al. 2018). Although prostatic artery embolization can provide benefit in patients with significant lower urinary tract symptoms, TURP appears to provide more clinically and statistically significant improvements in symptoms and quality of life. Prostatic artery embolization may result in fewer adverse events and side effects including patient reported erectile dysfunction (Zumstein V 2019). However, the procedure requires experienced radiologists since it is technically challenging with a large variation in prostatic arterial anatomy seen across patients.

Aquablation is performed using the Aquabeam Robotic System (Procept BioRobotics, Redwood City, CA) (Aljuri and Perkins, Minimally invasive devices for the treatment of prostate diseases 2016) and was approved by the FDA in 2017. Aquablation is recommended for symptomatic BPH patients with prostate sizes in the range of 30 to 80 grams. In a double-blind, multicenter, prospective, noninferiority trial comparing the safety and efficacy of Aquablation to TURP in 181 men with prostate sizes between 30 and 80 grams and moderate to severe lower urinary tract symptoms, Aquablation was non-inferior to TURP in efficacy, and superior to TURP in safety (Gilling P, WATER: a double-blind, randomized, controlled trial of Aquablation 2018). Over 3 years of treatment, improvements in International Prostate Symptom Scores were similar across both groups. Three-year BPH symptom reduction and urinary flow rate improvement were similar after Aquablation and TURP (Gilling P, Three-year outcomes after Aquablation therapy compared to TURP: results from a blinded, randomized trial 2020).

The Rezum system (Boston Scientific, Marlborough, MA) is a minimally invasive transuretheral water vapor therapy used to treat lower urinary tract symptoms secondary to BPH (Shadduck and Hoey 2013), (Hoey and Shadduck 2022). The Rezum system creates water vapor (steam) thermal energy through the application of radiofrequency current against an inductive coil heater in the device's handle. The steam (103° C.) can then be injected into the prostatic transition zone. Upon contact with prostatic tissue, the steam phase shifts or condenses from vapor to liquid, releasing and convectively delivering large amounts of thermal energy (540 calories/gram). This results in disruption of prostatic cellular membranes leading to immediate cell death and necrosis. MRI studies have demonstrated the ablative tissue was reduced in volume by 96% at 6 months after treatment. There was a mean reduction in whole prostate volume of 38% at 6 months (Das A 2020).

Checcucci et al. performed a study examining the perioperative and functional outcomes of recently introduced treatments for BPH, including Urolift, Rezum, temporary implantable nitinol device (Amparore D 2019), prostatic artery embolization, and intraprostatic injection (Checcucci, et al. 2021). They found that these new treatments can yield fast and effective relief of lower urinary tract symptoms without affecting patient quality of life. However, only Rezum, UroLift, and prostatic artery embolization had a minimal impact on patients' sexual function with respect to baseline, especially regarding preservation of ejaculation.

### Paclitaxel-Coated Balloon

Clinical studies on transurethral dilation of the prostate conducted in the 1990s showed that balloon dilation techniques were safe but long-term durability was a problem due to healing of the commissure, scar tissue formation, or general recovery of the initially compressed tissue (Donatucci C F 1993), (P. Reddy 1990). The Optilume® BPH Catheter System (Urotronic Inc., Plymouth, Minnesota, USA) is the first drug-coated balloon dilation system for the treatment of obstructive BPH (Wang, Barnett and Hendrickson, Drug-coated balloon catheters for body lumens 2022), (Wang and Barnett, Drug-coated balloon catheters for nonvascular strictures 2022), (Wang, Barnett and Hendrickson, Drug-coated balloon catheters for body lumens 2022).

The system combines mechanical dilation to achieve commissurotomy of the anterior prostatic urethra to open the urethral lumen, with the delivery of the drug paclitaxel approximately 5 minutes to the dilated area to maintain urethral patency. Clinical data from the EVEREST-I study showed that treatment for obstructive BPH with the Optilume BPH Catheter System provides clinically meaningful benefits, with rapid improvement in lower urinary tract symptom severity and voiding function within 2 weeks that were sustained through at least 2 years. The therapy also had an acceptable safety profile and improved quality of life (Kaplan, et al. 2021), (Pichardo M 2023).

The discussion above provides evidence that catheter techniques can be used to effectively treat BPH. Also, the data from the studies using local delivery of paclitaxel appear to be associated with very favorable clinical outcomes over at least 2 years. The device outlined below describes a more efficient delivery method of paclitaxel or other medicaments that can be used to treat BPH. In addition, since the device can be left in place for days to weeks instead of the 5 minutes (for the Optilume paclitaxel catheter), the prostatic tissue paclitaxel concentrations achieved with the proposed catheter will probably be high enough to effectively treat prostate cancer, especially for tumors located near the prostatic urethra.

### SUMMARY OF THE INVENTION

Embodiments of this invention overcome weaknesses of the prior art. Device and methods treat proliferative disorders such as BPH and other disorders, including cancer in organs other than the prostate.

Benefits over prior art include local medicament delivery; non-blocking of urine flow; selected directionality of medicament; and non-surgical destruction of tissue. Expected benefits include improved efficacy and fewer side effects.

One embodiment comprises a tubular, expandable metal mesh mounted onto a Foley catheter, which is held in place during treatment with a bladder balloon. The mesh is compressed for insertion, on the catheter, into the prostate. The mesh is then expanded so that at least a portion of the mesh is against the prostatic urethral wall. The mesh has been treated with a medicament, such as paclitaxel. Urine may freely flow through the catheter since there is no blockage of the urethra or the catheter lumen. Embodiments use different elements and methods for compressing or expanding the metal mesh. The mesh in a passive state may be either compressed or expanded, depending on the embodiment. The state of the mesh is changed by either pushing or pulling, from outside the body, on the mesh while it is in place in the urethra. Other embodiments may use the metal mesh as one electrode for iontophoresis or electroporation. Because the flow of urine is not blocked, treatment times may range from minutes to weeks.

### BRIEF DESCRIPTION OF DRAWINGS

FIG. 1 shows a simplified schematic of selected male human organs including a bladder, urethra, and prostate, for a patient with benign prostate hypertrophy.

FIG. 2 shows a Foley catheter placed in a patient.

FIG. 3 shows a mesh from FIG. 2 with the mesh in an expanded state.

FIG. 4A shows a deflated angioplasty balloon inside of a Foley catheter that has compressed mesh on the outer aspect of the Foley catheter.

FIG. 4B shows an angioplasty balloon inflated to expand the mesh.

FIG. 4C shows the angioplasty balloon of FIG. 4B deflated.

FIG. 4D shows the angioplasty balloon of FIG. 4C removed, leaving an expanded mesh.

FIG. 5 shows an embodiment with a semi-flexible tube adjacent to a compressed mesh. The distal segment of the mesh is bonded to the Foley catheter. When the semi-flexible catheter is advanced, the mesh is expanded.

FIG. 6 shows an embodiment with a compressed self-expanding mesh on the Foley catheter. The mesh is held in its compressed state by a semi-flexible catheter. When the semi-flexible catheter is withdrawn, the mesh expands. Advancing the semi-flexible catheter will compress the mesh back onto the Foley catheter.

FIG. 7 shows a schematic of an article of clothing adapted to perform as an electrode.

FIG. 8 shows a method of treatment that does not utilize a Foley catheter and allows for the patient to urinate normally during treatment.

FIG. 9 shows another element of a method of treatment.

### DETAILED DESCRIPTION OF INVENTION

Embodiments, descriptions, scenarios, and drawings are non-limiting. Descriptions below of a device and methods of use are exemplary scenarios.

All Figures are schematic, showing only key elements of embodiments and their methods of use.

Embodiments of the device and methods of treatment are used to treat proliferative disorders of the prostate gland, especially benign prostate hypertrophy (BPH). Embodiments may be used to treat prostate cancer. Embodiments may be used to treat organs other than a prostate, such as gall bladder, esophagus, tear ducts, and ovaries.

Prior art includes use of an expandable balloon coated with a medicament, such as paclitaxel.

Embodiments of the device use a tubular, expandable metal mesh, which is coated with or has embedded a medicament such as paclitaxel. A first improvement over prior art is that a higher dose of paclitaxel is possible than is possible using a balloon. A second improvement, since urine may flow in the urethra during treatment, is that an embodiment may be left in place for a longer treatment time-period than a balloon that occludes flow, such as minutes, hours, days, or weeks. Yet another improvement is that embodiments of a metal mesh permit more variation and specificity for treatment options than is available in prior art balloons. Higher effective doses of paclitaxel are feasible with embodiments, such that they may be used to treat cancers. Also, other medicaments may be incorporated onto the mesh. Yet more improvements include using the metal mesh as one electrode for iontophoresis or electroporation, or both. Yet another advantage is that the mesh may be further expanded, such as by steps or a ramp of expansion, during treatment. Medicament delivery is typically by passive diffusion, except when iontophoresis is used. The term, “distal,” herein, refers to a direction away from the external portion of a device. The term, “proximal,” herein, refers to a direction closer to the external portion of a device. Note that this usage may not be consistent with typical medical descriptions of body part locations. The mesh is conductive, not necessarily made of metal; although it is referred to as metal herein. The mesh is tubular; for brevity this adjective is not always used herein.

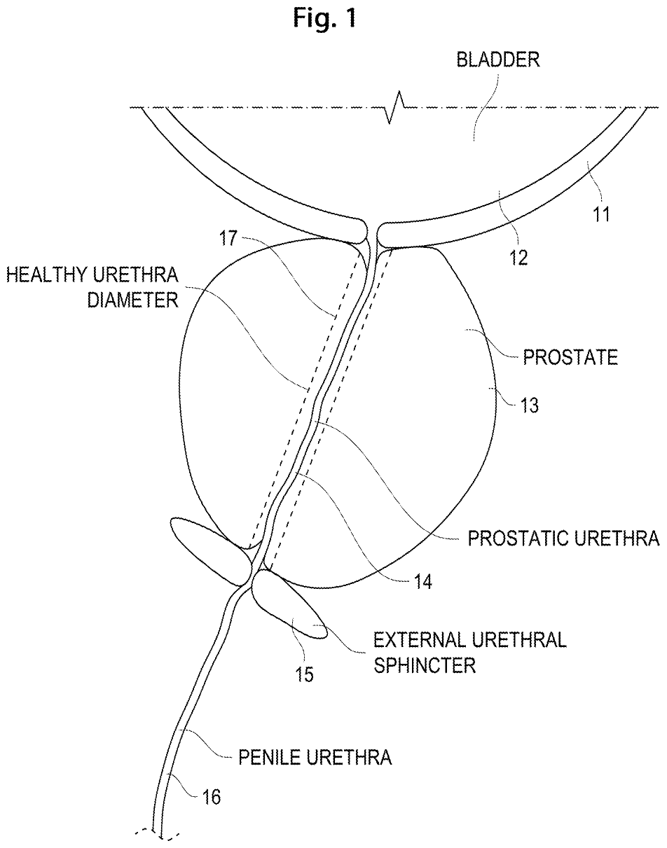

Turning first to FIG. 1, we see a schematic of a male patient with BPH, showing a cross-section of a portion of the bladder 12, bladder wall 11, two lobes of the prostate 13, a narrowed prostatic urethra 14, the external urethral sphincter muscle 15, and a portion of the penile urethra 16. The prostatic urethra 14 is shown narrowed, such as from benign prostate hypertrophy (BPH) or another pathology. The dotted line 17 shows a schematic, wider-diameter prostatic urethra such as might be in a healthy patient. FIG. 1, as well as other Figures, are not to scale. The narrowed prostatic urethra 14 may cause slow urination as well as other effects of BPH.

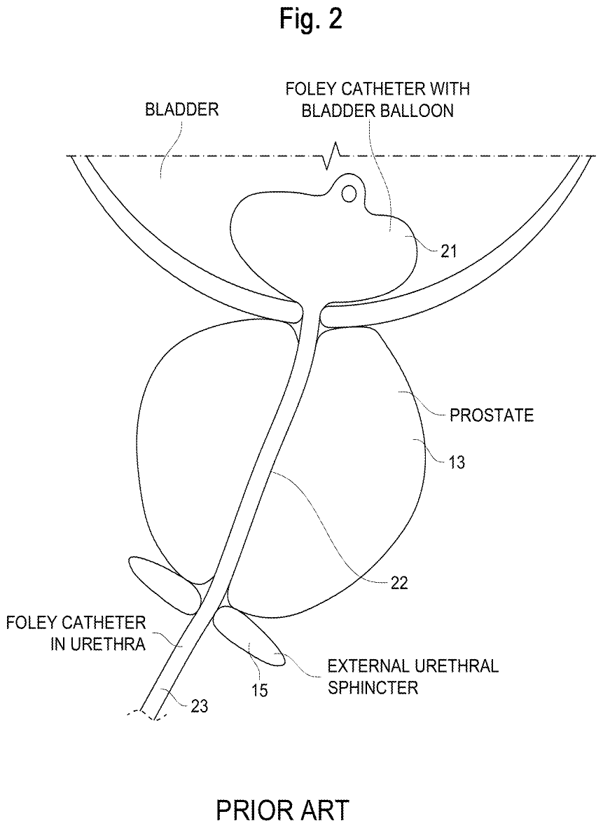

FIG. 2, prior art, shows a cross-section of prior art of a Foley catheter 23 inserted through the prostatic urethra 22 and held in place by an inflated balloon 21 in the bladder. Two lobes of the prostate are shown 13. The external urethral sphincter muscle is shown 15.

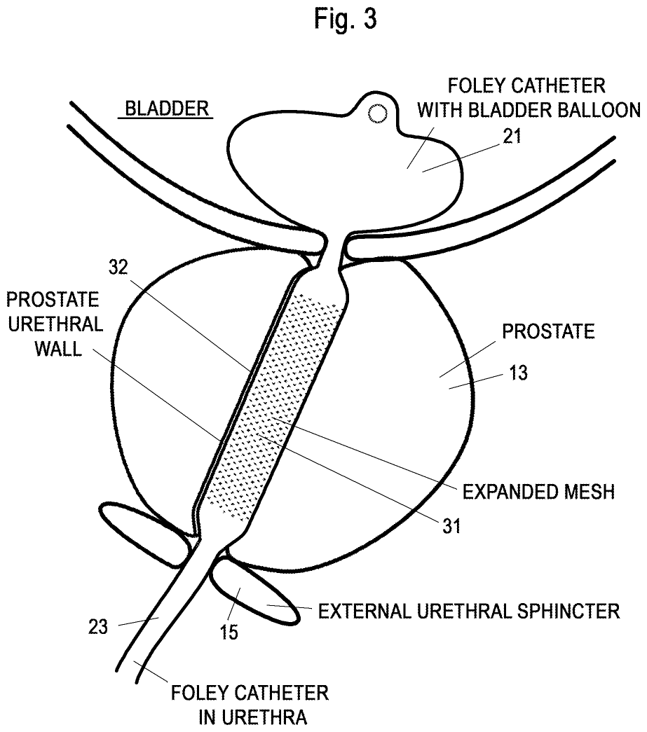

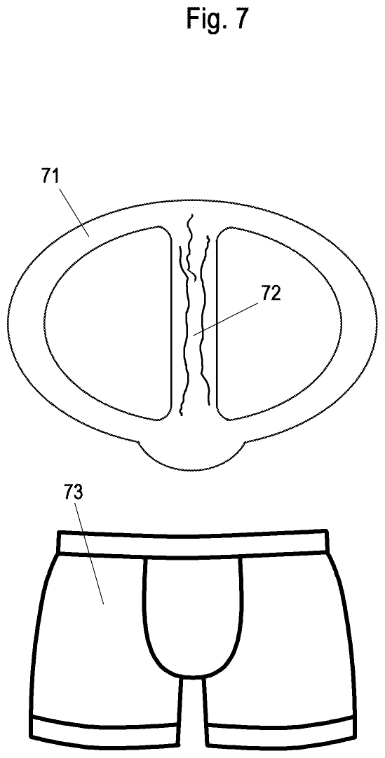

FIG. 3 shows one state of an embodiment using a tubular, expandable, metal mesh. The mesh generally has two states: compressed and expanded. Embodiments may use a range of intermediate expansions. Here, the mesh is shown schematically in an expanded state 31. This expanded state 31 of the mesh enlarges the adjacent portion of the prostatic urethra, shown schematically as a portion of the prostatic urethral wall 32. Generally, the expanded mesh 31 will be in close contact with at least a portion of the urethral wall 32. Expanded mesh may be made from metal or other conductive material, including hybrid materials such as a metal or conductive coating on a non-conductive material. This portion of the urethra may be referred to as a treatment site or treatment location. It may run the length of the prostate or may be shorter. The corresponding location on the outer catheter, or Foley catheter may be referred to as the treatment site or treatment location on the catheter. For treatment using ionophoresis or electroporation a conductive material such as a wire or a conductive portion of a catheter is used, extending from an electrical connection to the mesh to a proximal location outside the body. This wire is not shown. The wire is then connected to electronics, such as power supplies, signal generators, monitoring, and safety circuits, external to the patient, as known in the art. See also FIG. 7 for a schematic of a second electrode.

Generally, a treatment site for BPH is a portion of the prostatic urethra that is compressed or narrowed by the prostate gland.

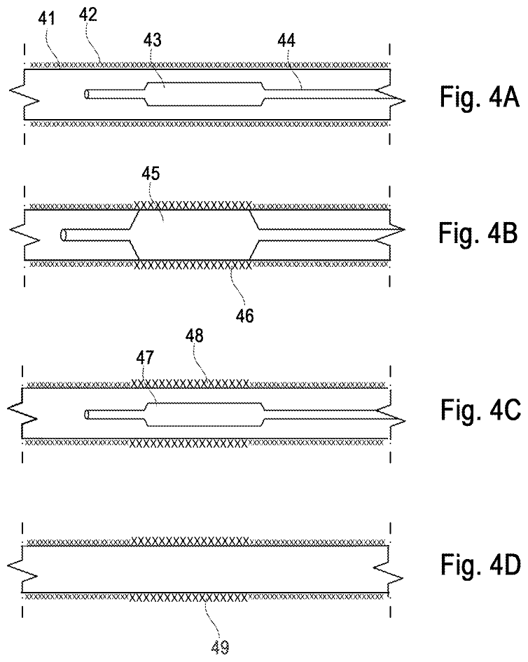

Turning now to FIGS. 4A, 4B, 4C and 4D, we see schematically cross-sections of steps in the placement and use of an expandable mesh. These figures and discussion are applicable to both device embodiments and method of treatment embodiments. 41 shows the walls of a Foley catheter placed inside a prostatic urethra. The prostatic urethra is not shown. See also FIG. 3. 42 shows a portion the mesh in a compressed state. The mesh 42 may be on the outside of the Foley catheter 41 or may be part of the Foley catheter 41. The mesh 42 may be effectively part of the structure of the outside catheter 41. 43 and 44 show portions of an inside catheter, placed inside of the Foley catheter 41. 43 shows a portion that is expandable, such as being flexible. It may function effectively as a balloon or angioplasty balloon. 44 shows a non-expanding region. FIG. 4A shows a state of a device embodiment or step in a treatment where the Foley catheter has been placed in a patient's prostatic urethra and the inside catheter 44 has been placed inside of the Foley catheter 41. The tubular mesh 42 may extend proximally to outside the patient. Embodiments may comprise a removable handle or clamp to secure the mesh at a desired location, such as a treatment location, on the outside catheter.

FIG. 4B shows a state of a device embodiment or step in a treatment where the flexible portion 43 of the inside catheter 44 has been expanded 45. This portion 43 presses against the compressed mesh 42 to force it outward towards, against, or into the prostatic urethra, not shown. This expands at least a portion 46 of the mesh. A drug such as paclitaxel or other treatment medicament now flows via passive diffusion or iontophoresis from the expanded mesh 46 into the surrounding prostate gland. The mesh contains an electrical connection that extends along or inside the Foley catheter that may connect via a wire, not shown, to external electronics such a power supply, signal generator, control, and safety electronics to implement ionophoresis or electroporation, or both, using the mesh 48 as one electrode.

FIG. 4C shows the inner catheter 44 returned to its non-expanded state, 47. The expanded mesh 48 remains expanded and in contact with the prostatic urethra, not shown.

FIG. 4D shows the inner catheter 44 removed from the outer catheter 41, leaving in place the expanded mesh 48. Drug or medicament flow from the expanded mesh 49 continues. Embodiments of the state of a device or step of treatment shown in FIG. 4D remain for an appropriate time-period of treatment, which may vary considerably, such as from a minute to weeks. Note that urine may still flow through the Foley catheter 41.

The mesh 42 may run from the prostatic urethra to outside the body. It may alternatively be shorter.

At the end of treatment, the expanded mesh 48 may be removed from the prostatic urethra by pulling it from a segment that extends outside the penis, compressing it to clear the inside of the urethral lumen. Then the Foley catheter may be removed, completing the use of the device or this stage of treatment. Alternatively, the Foley catheter 41 may be removed at the same time as the expanded mesh 49.

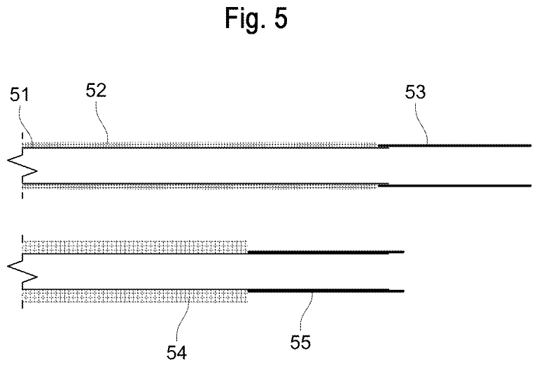

Turning now to FIG. 5, we see an embodiment that uses a semi-rigid catheter 53 that fits over the inner catheter 51 and does not fit over the compressed mesh 52. The distal segment of the mesh is bonded to the Foley catheter. The proximal segment of the mesh is attached to a semi-rigid catheter. Pushing the semi-rigid catheter 53 distally 55 compresses the mesh 54 longitudinally forcing it to expand radially, against the inner aspect of the prostatic urethra, where it can deliver medicament via passive or active diffusion into the prostate. Further, the metal or otherwise conductive mesh 54 may connect via a wire, not shown, to external electronics such a power supply, signal generator, control, and safety electronics to implement ionophoresis or electroporation, or both, using the mesh 54 as one electrode. Following treatment, the semi-rigid catheter is retracted, which contracts the mesh. Then, the bladder balloon 21 in FIG. 2. is deflated and the entire device is removed from the patient, completing a phase of treatment.

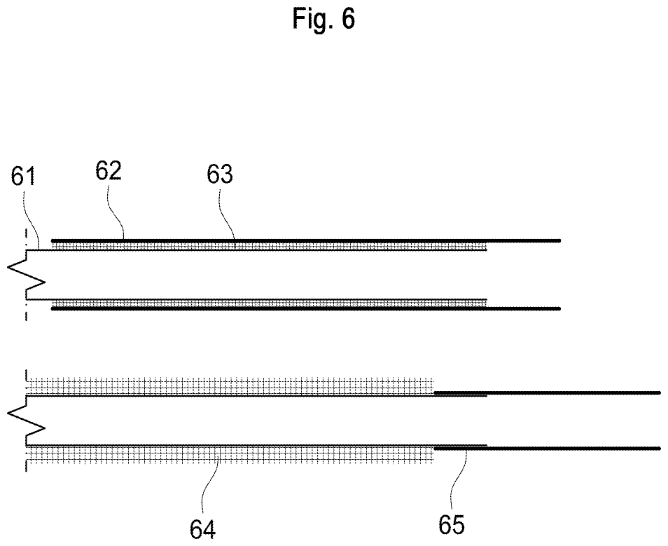

Turning now to FIG. 6, we see yet another embodiment. Here, a constraining catheter 62 is initially placed or extended distally, over the compressed, self-expanding mesh 63. The constraining catheter 62 holds the mesh 63 in its compressed state, attached to or part of the inner catheter 61. Then the constraining catheter 65 is withdrawn partially or fully, releasing the mesh 64 into its default or passive expanded state. Following treatment, the constraining catheter is advanced over the mesh, which returns it to its contracted state. The bladder balloon 21 in FIG. 2 is deflated and the entire device is removed from the patient, completing a phase of treatment.

Turning now to FIG. 7, we see an element of an embodiment that uses wearable clothing such as an undergarment 73, comprising electrode or electrodes 72. 73 shows a front view of appropriate, specially made briefs. 71 shows a transverse view of the undergarment where electrode wires are placed. Such wires may be on the inner surface of the undergarment or incorporated into the cloth. Wires 72 may be any suitably conductive and at least semi-flexible material. The wires 72 are connected (not shown) to external electronics such a power supply, signal generator, control, and safety electronics, not shown. The conductive wires 72 make up at least part of a second electrode, that in conjunction with a first electrode, the metal or otherwise electrically conductive mesh, such as 31 in FIG. 3, 49 in FIG. 4, 54 in FIG. 5, or 64 in FIG. 6 that form the two electrodes necessary for ionophoresis or electroporation, or both. Such device elements and treatment may cause a medicament in the expanded mesh to move preferentially into the prostate, 31 in FIG. 3. Such preferential movement of medicament may include faster diffusion, deeper diffusion, longer treatment time, and directional diffusion. Such preferential movement is particularly advantageous when treating a tumor.

A medicament in the metal may be held in place by various methods for different embodiments and variations in treatment. On one embodiment the medicament is, or is dissolved in or suspended in, a liquid, gel, hydrogel, foam or paste. Once in place for treatment, this medicament and any medicant carrier melts, dissolves, or diffuses into the tissue being treated. The medicament may be held in place initially with surface tension, friction, pressure, and the like. In another embodiment the metal mesh is treated or formed with a texture pattern that assists in retaining the medicament or its carrier. In another embodiment the metal mesh is first coated with a substance upon which the medicament or its carrier prefers to stick or bond. In yet another embodiment, a mesh with a medicament is coated with a removable or dissolvable substance, once in place for treatment, for example, a gelatin or a fat that melts below body temperature. In yet another embodiment, the mesh and the medicament are covered with a sheath. Once in place, the sheath is removed (such as by a thin cable), exposing the medicament. In another embodiment a rigid material is suffused with holes. The medicant is held temporarily with surface tension in the holes. Once in place for treatment the medicant slowly dissolves. Thus, in the context of embodiments, the phrase “medicant in a metal mesh,” and “medicament on a metal mesh” are similar enough that they should be construed as functionally equivalent, unless otherwise indicated.

Embodiment descriptions may refer to a “metal mesh.” Although this is preferred, many other materials may be used, such as plastics, which typically, in these embodiments, would be semi-rigid such that the mesh may readily be deformed from a compressed state to an expanded state. A mesh material that is non-metallic should be construed as equivalent, if for the purposes of an embodiment, it functions substantially similarly. Non-metallic material will not typically function as an electrode for ionophoresis or electroporation embodiments because it is typically non-conductive. However, some non-metallic materials are sufficiently conductive, or have been modified or coated to make them sufficiently conductive. Note with the relatively low currents used with ionophoresis and electroporation conductive materials may be used that are minimally conductive and yet sufficiently conductive for use as an electrode for ionophoresis and electroporation applications.

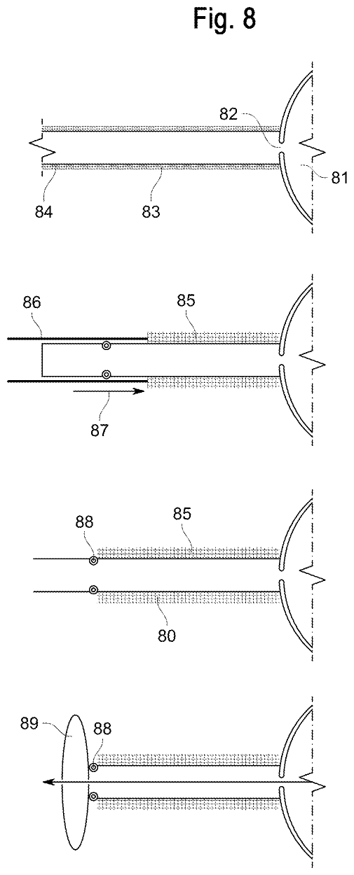

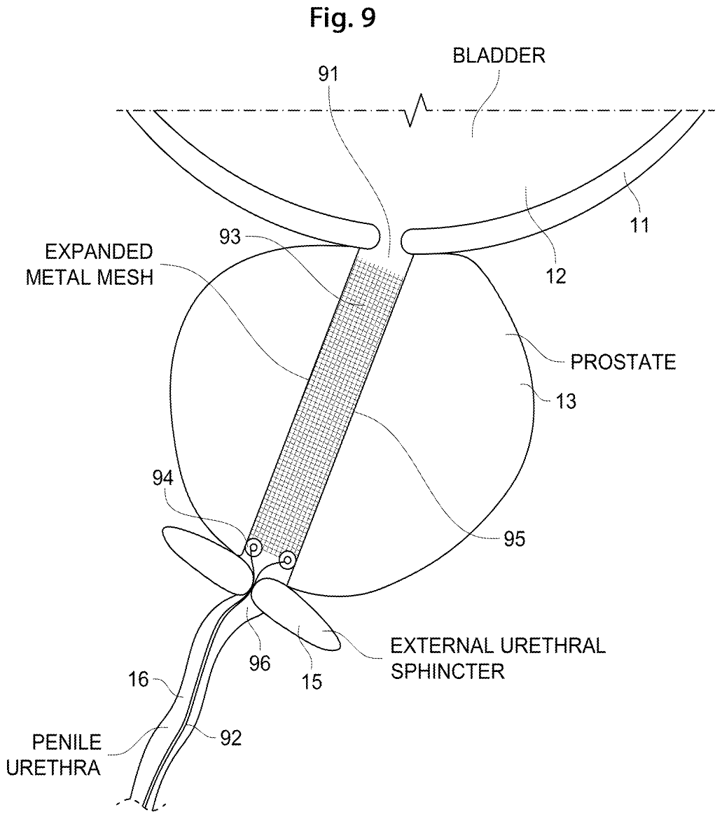

Turning now to FIG. 8 we see illustrated schematically yet additional embodiments. These embodiments have the benefit to the patient and the treatment as permitting the patient to urinate normally and voluntarily during a treatment period. This is not only more comfortable for the patient, but it also permits significantly longer treatment times, such as hours, days, or months. Embodiments in FIGS. 8 and 9 comprise a semi-rigid catheter 86 which is used temporarily to change the state of expandable metal mesh from compressed 83 to expanded 85. In one simple embodiment the expandable metal mesh 83 is “pushed” (in the distal direction) to so change its state, as shown in 85. Note that in this context, “compressed” means a diameter of the mesh small enough that it may be inserted into the prostatic urethra; while “expanded” means the diameter of the mesh is radially larger such that the mesh stays in place, and indeed may press against the inside of the prostatic urethra. Expanded and compressed do not refer to an axial dimension. Indeed, in some embodiments the radial diameter and the axial length of the mesh change with opposite signs. “Pushing” on the mesh shortens the axial length—see the change from 83 to 85—while expanding radially.

FIG. 8 shows, in four stages, both simplified device embodiments and a simplified method of treatment. Stage I is at the top of FIG. 8, sequencing through to Stage IV at the bottom of FIG. 8. Not shown in FIG. 8 is patient anatomy such as the prostate and the prostatic urethra. 81 is a portion of the bladder with the internal urethral orifice 82. 84 is a portion of catheter placed inside the prostatic urethra, where the expandable metal mesh 83 is on the outside of the catheter 84. The catheter 84 may change shape or diameter at the location the expandable mesh 83. In this way, the combined diameter of the catheter 84 and the compressed metal mesh 83 may be comparable, which may aid in placement. Urine flow in FIG. 8 is typically from right (the bladder) to the left (penile urethra, not shown). To expand the mesh to its expanded state 85 a semi-rigid catheter 86 is placed outside of the catheter 84 (inside the prostatic urethra, not shown) and pushed distally until it contacts the metal mesh 85. The semi-rigid catheter 86 is then advanced, shown as arrow 87, pushing on the mesh 85, to change its state from compressed 83 to expanded 85, wherein the mesh then presses against the inside of the prostatic urethra, not shown. The semi-rigid catheter 86 is then removed, leaving the mesh in its expanded state 85. The catheter 84 comprises an operable junction shown schematically as 88. Such an operable junction may be a screw fit or a separable perforation. The catheter 84 is then separated into two portions at the operable junction 88. The proximal portion is then removed, leaving the distal portion and the expanded mesh in place in the patient's prostatic urethra. It is important in these embodiments that when the proximal portion of the catheter 84 is removed, the remaining expanded mesh 85 and the distal portion of the catheter have appropriate dimensions, such as length, so that the external urethral sphincter muscle 89 is not blocked. Typically, the internal urethral orifice 82 is also free of blockage. In this way, the patient may urinate normally and voluntarily, in the direction of the large arrow, such as to void the bladder, by the normal use of the external urethral sphincter muscle 89. In these embodiments, the treatment time with mesh 85 in place in the patient's prostate may be hours, days, weeks, or months. In some embodiments, a balloon with at least one orifice for urine flow may be located inside the bladder to hold the expanded mesh 85 in place. In such embodiments, another lumen in the catheter 84 may be used to inflate and deflate the balloon (not shown).

FIG. 8 shows an optional conductive wire, string, or cable 80, electrically attached to the expanded conductive mesh 85. In this way, in some embodiments, the expanded conductive mesh 85 may be used as one electrode for ionophoresis or electroporation. See also FIG. 7. The wire, string, or cable 80 may also be used, in some embodiments to remove or assist in the removal or the expanded metal mesh 85 (or compressed 83) and catheter portion 92 at the end of a treatment time. In yet other embodiments a separate wire, string or cable may be used for removal. In some embodiments, the expanded mesh 85 may be changed to a compressed state 83 prior to removal. A semi-rigid catheter 86 (or similar to 86) may be used for this purpose.

Turning now to FIG. 9, we see elements from FIG. 8 shown in a different view. Here a portion of the bladder wall 11 is shown, along with a portion of the bladder 12. The internal urethral orifice is shown 91. The prostate gland 13 is shown in schematic cross-section. The external urethral sphincter muscle is also shown in a schematic cross-section 15. A portion of the penile urethra is shown 16. The prostatic urethra is shown 95, with catheter 92 inside. The Key to embodiments is the expanded metal mesh 93 and a distal portion of a catheter 92 remaining inside the prostatic urethra 95 during treatment, while at the same time not blocking normal urine flow through prostate external opening 96 at the external urethral sphincter 15. Normal urine flow is from the bladder 12, through a portion of the catheter 92, then through the external urethral sphincter 15, then out through the penile urethra 16. During at least some of the treatment time the patient may use his external urethral sphincter 15 to voluntarily control urine flow. Schematic operatable junction point 94 is shown, here with the proximal portion of the catheter 92 removed.

In some embodiments, magnets or electromagnets, or both, may be used to assist changing the state of the metal mesh (83, 85), or placement or removal of the metal mesh, or separation of the catheter 84 into a distal and proximal portion at junction 88, in any combination. Use of such magnets or electromagnets may be external to patient's body or internal in the catheter 84, in any combination.

Methods of treatments as embodiments typically use the elements as described above and shown in FIG. 1 through FIG. 9.

One such treatment embodiment comprises placing an expandable, tubular mesh such as 31 (shown in expanded state in FIG. 3) into a urethra in the prostate gland 13 to a treatment site in the prostate gland, such as the length of prostatic urethra between the external bladder orifice 91 and the external sphincter muscle 15. In this and similar embodiments the expandable tubular mesh 31, 85, 42, 46, 93 comprises a medicament for treatment of the prostate, such as paclitaxel. An angioplasty balloon 21 is placed inside the mesh in the prostatic urethra and expanded. The expandable tubular mesh is changed from its compressed state to its expanded state, where it presses against the inside of the prostatic urethra at the treatment site. The angioplasty balloon is then deflated and removed, leaving the expandable metal mesh in the urethra at the treatment site. Urine may then flow unimpeded from the bladder through the penis. After the treatment time, the expandable metal mesh is contracted and removed.

In yet another treatment embodiment, the tubular expandable metal mesh comprises a semi-rigid catheter attached at a proximal end of the expandable metal mesh and is pushed against the mesh to change it from its compressed state to its expanded state.

In yet another embodiment, a self-expanding tubular metal mesh is inside a separable sheath. The separable sheath is pulled causing the expandable tubular metal mesh to change from its compressed state to its expanded state. Urine may then flow unimpeded from the bladder through the penis. After the treatment time, the sheath is advanced over the mesh the expandable metal mesh, which causes it to contract, and is removed.

In yet another embodiment, the expandable, tubular mesh surrounds a portion of a urinary catheter; this mesh is placed into a treatment site in the prostatic urethra. Steps of the above methods are then followed.

In yet another embodiment, an expandable tubular mesh is placed into a urethra in the prostate gland to a treatment site. The tubular mesh is attached to at least a portion of a urinary catheter, wherein the urinary catheter comprises two portions: a proximal removable portion and a distal semi-permanent portion. The two portions are then separated. A removable portion is then removed, leaving the other portion in place. A semi-rigid catheter is then placed in the patient's urethra; the semi-rigid catheter is then pushed to change the state of the tubular, expandable metal mesh from its compressed state to its expanded state; the semi-rigid catheter is then removed. During a treatment time, the patient may voluntarily use his external urinary sphincter to control the flow of urine from his bladder, through the expandable tubular mesh into the penile urethra. At the end of the treatment time the expandable tubular mesh is contracted and removed from the urethra, along with the distal portion semi-permanent portion of the urinary catheter.

Additional method embodiments use combinations of the above method steps.

Method claims that refer to a device claim are construed where the reference to the embedded device claim may be replaced by exactly the elements and other limitations in the device claim. Device claims that refer to a method claim are construed where the embedded method claim may be replaced by exactly the steps and other limitations if the method claim.

In most contexts, the phrases, “improvement over the prior art,” and “advantages over the prior art” are equivalent. There are obvious overlaps between a device and a method of use of the device, that is, a treatment of a patient. Terms, descriptions, and Figures should be construed for either a device or a method of use, or both, as appropriate.

Although the term, “metal” is used herein, functional alternatives to metal are suitable for some embodiments and “metal” shall be construed to includes such functional alternatives. A material may be made electrically conductive by means of a conductive coating, conductive layer, or embedding a conductive material within a non-conductive material. An advantage of metal or other conductive elements is that the mesh may be used as one electrode for iontophoresis, or electroporation, or both. For devices, methods of use and treatment that do not require iontophoresis or electroporation a non-conductive mesh may be used and a wire extending from the mesh to outside the patient is not necessary.

References herein to urethra and prostate are non-limiting both for a device and a method of treatment. Lumens other than a urethra may be used. Embodiments of the device may be used for treatment of organs, glands, or lumens other than a prostate.

Ideal, Ideally, Optimal and Preferred—Use of the words, “ideal,” “ideally,” “optimum,” “optimum,” “should” and “preferred,” when used in the context of describing this invention, refer specifically a best mode for one or more embodiments for one or more applications of this invention. Such best modes are non-limiting and may not be the best mode for all embodiments, applications, or implementation technologies, as one trained in the art will appreciate.

All examples are sample embodiments. In particular, the phrase “invention” should be interpreted under all conditions to mean, “an embodiment of this invention.” Examples, scenarios, and drawings are non-limiting. The only limitations of this invention are in the claims.

May, Could, Option, Mode, Alternative and Feature—Use of the words, “may,” “could,” “option,” “optional,” “mode,” “alternative,” “typical,” “ideal,” and “feature,” when used in the context of describing this invention, refer specifically to various embodiments of this invention. Described benefits refer only to those embodiments that provide that benefit. All descriptions herein are non-limiting, as one trained in the art appreciates.

Embodiments of this invention explicitly include all combinations and sub-combinations of all features, elements, and limitation of all claims. Embodiments of this invention explicitly include all combinations and sub-combinations of all features, elements, examples, embodiments, tables, values, ranges, and drawings in the specification and drawings. Embodiments of this invention explicitly include devices and systems to implement any combination of all methods described in the claims, specification, and drawings. Embodiments of the methods of invention explicitly include all combinations of dependent method claim steps, in any functional order. Embodiments of the methods of invention explicitly include, when referencing any device claim, a substation thereof to any and all other device claims, including all combinations of elements in device claims. Claims for devices and systems may be restricted to perform only the methods of embodiments or claims.

### BIBLIOGRAPHY

Aljuri, Nikolai, and Rodney Perkins. 2016. Minimally invasive devices for the treatment of prostate diseases. U.S. Pat. No. 9,364,250 B2. June 14.
Aljuri, Nikolai, Surag Mantri, Luis Baez, George Surjan, Michael Sasnett, and Jonathan Foote. 2017. Tissue resection and treatment with shedding pulses. U.S. Pat. No. 9,848,904 B2. December 26.
Amparore D, De Cillis S, Volpi G, et al. 2019. “First-and second-generation temporary implantable nitinol devices as minimally invasive treatments for BPH-related LUTS: systematic review of the literature.” Current Urolology Reports 20: 47.
Berry S J, Coffey D S, Walsh P C, Ewing L L. 1984. “The development of human benign prostatic hyperplasia with age.” J Urology 132 (3): 474-479.
Bolmsjo, Magnus, and Sonny Schelin. 2003. Method and device for the treatment of prostate tissue. U.S. Pat. No. 6,524,270 B1. February 25.
Catanese, Joseph, Theodore Lamson, Joshua Makower, Amik Nagpurkar, Amrish Walke, Claude Vidal, Russell, Collinson, Michael Redmond, and Jacqueline Welch. 2010. Devices, systems and methods for retracting, lifting, compressing, supporting or repositioning tissue or anatomical structures. U.S. Pat. No. 7,645,286 B2. January 12.
Checcucci, Enrico, Alessandro Veccia, Sabrina De Cillis, Federico Piramide, and Gabriele, et al. Volpi. 2021. “New ultra-minimally invasive surgical treatment for benign prostatic hyperplasia: a systematic review and analysis of comparative outcomes.” European Urology Open Science 33: 28-41.
Cheng, Floria, Michael Gearhart, Johnson Andrew, and et al. 2013. Anchor delivery system. USA Patent 20130096582A1. April 13.
Committee, AUA Practice Guidelines. 2003. “AUA guideline on management of benign prostatic hyperplasia (2003). Chapter 1: Diagnosis and treatment recommendations.” J Urology 170: 530-547.
Condon, Dennis, Robert Bley, and Bobby Purkait. 1993. Transparent prostate dilation balloon and scope. U.S. Pat. No. 5,188,596. February 23.
Connolly S S, Fitzpatrick J M. 2007. “Medical treatment of benign prostatic hypertrophy.” Postgraduate Medical Journal 976: 73-78.
Das A, Han T M, Uhr A, et al. 2020. “Benign prostatic hyperplasia: an update on minimally invasive therapy including Aquablation.” Canadian Journal of Urology 27 (Int Suppl 2): 2-10.
Donatucci C F, Berger N, Kreder K J, Donohue R E, Raife M J, Crawford E D. 1993. “Randomized clinical trial comparing balloon dilatation to transurethral resection of prostate for benign prostatic hyperplasia.” Urology 42: 42-49.
Fenn, Alan, and John Mon. 2022. System and method for heating the prostate gland to treat and prevent the growth and spread of prostate tumors. U.S. Pat. No. 6,477,426 B1. November 5.
Fitzpatrick, M. 2006. “The natural history of benign prostatic hyperplasia.” British Journal of Urology International 97 (Supplement 2): 3-6.
Foster H E, Dahm P, Kohler T S, Lerner L B, Parsons J K, Wilt T J, et al. 2019. “Surgical management of lower urinary tract symptoms attributed to benign prostatic hyperplasia: AUA Guideline Amendment 2019.” Journal of Urology 202: 592-598.
Fukuta F, Masumori N, Mori M, Tsukamoto T. 2012. “Natural history of lower urinary tract symptoms in Japanese men from a 15-year longitudinal community-based study.” British Journal of Urology International 110 (7): 1023-1029.
Gilling P, Barber N, Bidair M, et al. 2020. “Three-year outcomes after Aquablation therapy compared to TURP: results from a blinded, randomized trial.” Canadian Journal of Urology 27 (1): 10072-10079.
Gilling P, Barber N, Bidair M, et al. 2018. “WATER: a double-blind, randomized, controlled trial of Aquablation.” Journal of Urology 199 (5): 1252-1261.
Gratzke C, Schlenker B, Seitz M, et al. 2007. “Complications and early postoperative outcome after open prostatectomy in patients with benign prostatic enlargement: results of a prospective multicenter study.” Journal of Urology 177: 1419-1422
Hargreave T B, McNeill A S. 2005. “Acute urinary retention in men: the risks and outcomes with medical therapy.” Current Urology Reports. 6: 263-270.
Hoey, Michael, and John Shadduck. 2022. Systems and methods for prostate treatment. USA Patent 2022/0233228 A1. July 28.
Kaplan, Steven, Marycarla Pichardo, Edswin Rijo, Gustavo Espino, Ramon Rodriguez Lay, and Rafael Estrella. 2021. “One-year outcomes after treatment with a drug-coated balloon catheter system for lower urinary tract symptoms related to benign prostatic hyperplasia.” Prostate Cancer and Prostatic Diseases 24: 1073-1079.
Miernik, Arkadiusz, and Christian Gratzke. 2020. “Current treatment for benign prostatic hyperplasia.” Dtsch Arztebl Int 117 (49): 843-854.
Pichardo M, Rijo E, Espino G, Lay R R, Estrella R, Gonzalez C, Fernandez M, Soriano D, Peralta I M, Kaplan S A. 2023. “Durable benefit after treatment of obstructive benign prostatic hyperplasia with a novel drug-device combination product: 2-year outcomes from the EVEREST-I study.” World Journal of Urology 41 (8): 2209-2215.
Raiche, Paul. 1943. Catheter. U.S. Pat. No. 2,320,157. May 25.
Reb, Phillipe, Marion Pierre, Barbara De Gioannis, and James Krom. 2018. Microspheres useful for therapeutic vascular embolization. U.S. Pat. No. 10,022,469 B2. July 17.
Reddy, P K. 1990. “Role of balloon dilation in the treatment of benign prostatic hyperplasia.” Prostate Supplement 3: 39-48.
Reddy, Pratap, Michael Mikulich, and David Clark. 1990. Prostate balloon dilator. U.S. Pat. No. 4,932,956. June 12.

Reich O, Gratzke C, Bachmann A, et al. 2008. “Morbidity, mortality and early outcome of transurethral resection of the prostate: a prospective multicenter evaluation of 10,654 patients.” Journal of Urology 180: 246-249.

Roehrborn C, Barkin J, Gange S N, et al. 2017. “Five year results of the prospective randomized controlled prostatic urethral LIFT study.” Canadian Journal of Urology 24: 8802-8813.

Rosenbluth, Robert. 1990. Method and apparatus for treating hypertrohy of the prostate gland. U.S. Pat. No. 4,893,623. January 16.

Rosenbluth, Robert. 1988. Method and apparatus for treating hypertropy of the prostate gland. U.S. Pat. No. 4,762,128. August 9

Scott, Neal. 2020. Catheter for localized drug delivery. U.S. Pat. No. 10,661,055 B1. May 26.

Scott, Neal, Terry Burkoth, and Scott Harris. 2005. High concentration medicament and polymer coated device for passive diffusional medicament delivery. USA Patent 2005/0159704 A1. July 21.

Shadduck, J H, and M Hoey. 2013. Systems and methods for treatment of prostatic tissue. U.S. Pat. No. 8,388,611 B2. March 5.

Sterzer, Fred. 1991. Catheters for treating prostate disease. U.S. Pat. No. 5,007,437. April 16.

Swoyer, John, and Thomas Skwarek. 2007. Device and method for transurethral prostate treatment. U.S. Pat. No. 7,238,182 B2. July 3.

Wang, Lixiao, and Peter Barnett. 2022. Balloon catheters for body lumens. U.S. Pat. No. 11,439,801 B2. September 13.

Wang, Lixiao, and Peter Barnett. 2022. Drug coated balloon catheters for non vascular strictures. U.S. Pat. No. 11,484,628 B2. November 1.

Wang, Lixiao, and Peter Barnett. 2022. Drug-coated balloon catheters for nonvascular strictures. U.S. Pat. No. 11,484,628 B2. November 1.

Wang, Lixiao, Peter Barnett, and David Hendrickson. 2022. Drug coated balloon catheters for body lumens. U.S. Pat. No. 11,504,450. November 22.

Wang, Lixiao, Peter Barnett, and David Hendrickson. 2022. Drug-coated balloon catheters for body lumens. U.S. Pat. No. 11,471,656 B2. October 18.

Wang, Lixiao, Peter Barnett, and David Hendrickson. 2022. Drug-coated balloon catheters for body lumens. U.S. Pat. No. 11,504,450 B2. November 22.

Young M J, Elmussareh M, Morrison T, Wilson J R. 2018. “The changing practice of transurethral resection of the prostate.” Annals Royal College of Surgery (England) 100: 326-329.

Zhang Y, Yuan P, Ma D, et al. 2019. “Efficacy and safety of enucleation vs resection of prostate for treatment of benign prostatic hyperplasia: a meta-analysis of randomized controlled trials.” Prostate Cancer Prostatic Dis. 22: 493-508.

Zumstein V, Betschart P, Vetterlein M W et al. 2019. “Prostatic artery embolization versus standard surgical treatment for lower urinary tract symptoms secondary to benign prostatic hyperperplasia: a systematic review and meta-analysis.” European Urology Focus 5 (6): 1091-1100.

We claim:

1. A device for treatment of proliferative disorders of the prostate gland comprising:
an expandable tubular mesh comprising:
a compressed state and an expanded state;
a treatment medicament;
wherein the expandable tubular mesh is adapted to be inserted into a urethra;
a urinary catheter adapted to be inserted into a urethra via a penis;
wherein the expandable tubular mesh is attached to a portion of the urinary catheter;
wherein the length of the expandable mesh is adapted to extend axially from a treatment site in the urethra to a locking site external to the penis;
an angioplasty balloon adapted to be inserted into the urinary catheter to the treatment site;
wherein the expandable tubular mesh is adapted to expand from the compressed state to the expanded state upon inflation of the angioplasty balloon.

2. The device of claim 1 further comprising:
an electrical wire attached to the expandable tubular mesh;
wherein the expandable tubular mesh is at least partially electrically conductive;
wherein a proximal end of the electrical wire extends outside the penis when at least a portion of the expandable tubular mesh is at the treatment site.

3. A device for treatment of proliferative disorders of the prostate gland comprising:
an expandable tubular mesh comprising:
a compressed state and an expanded state;
a treatment medicament;
wherein the expandable tubular mesh is adapted to be inserted into a urethra;
a urinary catheter adapted to be inserted into a urethra via a penis;
wherein a distal end of the expandable tubular mesh is attached to a first portion of the urinary catheter;
a semi-rigid catheter adapted be placed in the urethra surrounding a portion of the urinary catheter;
wherein a proximal end of the expandable tubular mesh is adapted to mate with a distal end of the semi-rigid catheter;
wherein the expandable tubular mesh is adapted to change from the compressed state to the expanded state due to movement of the semi-rigid catheter axially along the urinary catheter.

4. The device of claim 3 further comprising:
an electrical wire attached at a distal end to the expandable tubular mesh;
wherein the expandable tubular mesh is at least partially electrically conductive;
wherein a proximal end of the electrical wire extends outside the penis when the expandable tubular mesh is at the treatment site.

5. A device for treatment of proliferative disorders of the prostate gland comprising:
an expandable tubular mesh comprising:
a compressed state and an expanded state;
a treatment medicament,
wherein the expandable tubular mesh is adapted to be inserted into a urethra;
a urinary catheter adapted to be inserted into a urethra via a penis;
wherein a proximal end of the expandable tubular mesh is attached to a first portion of the urinary catheter;
a restraining catheter adapted be placed in the urethra surrounding a portion of the urinary catheter and a treatment portion of the expandable tubular mesh when the expandable tubular mesh is in the compressed state;
wherein the restraining catheter is adapted to be removed from the treatment portion of the expandable tubular mesh when the restraining catheter is moved axially;
wherein the treatment portion of the expandable tubular mesh is adapted to change from the compressed state to the expanded state when the restraining catheter is removed from the treatment portion of the expandable tubular mesh.

6. The device of claim 5 further comprising:
an electrical wire attached at a distal end to the expandable tubular mesh;
wherein the expandable tubular mesh is at least partially electrically conductive;
wherein a proximal end of the electrical wire extends outside the penis when the expandable tubular mesh is at the treatment site.

7. The device of claims 2, 4 or 6 further comprising:
a garment adapted to be worn by a patient comprising an electrically conductive portion adapted to make electrical contact with the patient's skin;
wherein the garment, when worn by the patient, is adapted to be a first electrode for iontophoresis or electroporation, or both, and
wherein a second electrode for the iontophoresis or electroporation, or both, comprises at least a portion of the expandable tubular mesh.

8. A device for treatment of proliferative disorders of the prostate gland of a patient comprising:
an expandable tubular mesh comprising:
a compressed state and an expanded state;
a treatment medicament,
wherein the expandable tubular mesh is adapted to be inserted into a urethra;
a urinary catheter adapted to be inserted into a urethra via a penis;
wherein a proximal end of the expandable tubular mesh is attached to a first portion of the urinary catheter;
wherein the urinary catheter comprises a urine lumen and a balloon inflation lumen;
wherein the balloon inflation lumen is adapted to inflate a balloon inside a patient's bladder;
wherein the urinary catheter comprises to portions: a proximal removable portion and a distal semi-permanent portion;
wherein the proximal removable portion is adapted to be detached from the distal semi-permanent portion, leaving the distal semi-permanent portion in the prostatic urethra;
a semi-rigid removable catheter adapted to be inserted into the patient's penis extending from outside the patient to the proximal end of the expandable tubular mesh;
wherein the semi-rigid removable catheter is adapted to press against the proximal end of the proximal end of the expandable tubular mesh causing the expandable tubular mesh to expand against the inside of the prostrate urethra; and
wherein the semi-rigid removable catheter is adapted to be removed from the patient during a treatment time.
wherein the expandable tubular mesh and the semi-permanent portion of the urinary catheter are adapted to remain in the patient's prostatic urethra during the treatment period and during the treatment period the expandable tubular mesh and the semi-permanent portion of the urinary catheter are free of any proximal or distal portion inside of the patient's external urinary sphincter; and
wherein during at least a portion of the treatment period the patient is able to voluntarily use his external urinary sphincter to control the flow of urine from the bladder, through the expandable tubular mesh into the penile urethra.

9. The device of claim 8 further comprising:
an electrical wire extending from the expandable tubular mesh, through the patient's penile urethra, to the outside of the patient's body;
wherein the electrical wire is adapted to be used with ionophoresis or electroporation;

10. A method of treatment of proliferative disorders of the prostate gland comprising the steps:
placing an expandable, tubular mesh into a urethra in the prostate gland to a treatment site,
wherein the expandable tubular mesh comprises a compressed state and an expanded state,
wherein the expandable tubular mesh comprises a treatment medicant;
placing an angioplasty balloon inside the expandable tubular mesh;
inflating the angioplasty balloon to change the state of the expandable tubular mesh from the compressed state to the expanded state,
wherein the expanded tubular mesh presses against the inside of the urethra;
removing the angioplasty balloon;
wherein the expandable tubular mesh remains in the urethra at the treatment site after the removal of the angioplasty balloon,
wherein urine may flow unimpeded from a bladder through the urethra after the removal of the angioplasty balloon;
waiting a treatment time;
wherein the treatment medicament diffuses into the prostate gland during the treatment time; and
removing the expandable tubular mesh from the urethra.

11. A method of treatment of proliferative disorders of the prostate gland comprising the steps:
placing an expandable, tubular mesh into a urethra in the prostate gland to a treatment site,
wherein the expandable tubular mesh comprises a compressed state and an expanded state,
wherein the expandable tubular mesh comprises a medicament,
wherein the expandable tubular mesh comprises a semi-rigid catheter attached a proximal end of the expandable tubular mesh,
pushing the semi-rigid catheter, wherein the pushing causes the expandable tubular mesh to change from the compressed state to the expanded state;
wherein the expanded tubular mesh presses against the inside of the urethra; and
removing the expandable tubular mesh.

12. A method of treatment of proliferative disorders of the prostate gland comprising the steps:
placing an expandable, tubular mesh into a urethra in the prostate gland of a patient to a treatment site,
wherein the expandable tubular mesh comprises a compressed state and an expanded state,
wherein the expandable tubular mesh comprises a medicament,
wherein the expandable tubular mesh is inside a separable sheath;
pulling the separable sheath away from the expandable tubular mesh,
wherein the pulling causes the expandable tubular mesh to change from the compressed state to the expanded state;
wherein the expanded tubular mesh presses against the inside of the urethra; and
allowing the patient to urinate freely through the expandable tubular mesh free of blockage of the urethra;
waiting a treatment time;
removing the expandable tubular mesh.

13. A method of treatment of proliferative disorders of the prostate gland of a patient comprising the steps:
placing an expandable, tubular mesh surrounding a portion of a urinary catheter, into a urethra in the prostate gland of the patient to a treatment site;
wherein the expandable tubular mesh comprises a compressed state and an expanded state,
wherein the expandable tubular mesh comprises a medicament,
pulling the separable sheath away from the expandable tubular mesh,
wherein the pulling causes the expandable tubular mesh to change from the compressed state to the expanded state;
wherein the expanded tubular mesh presses against the inside of the urethra; and
allowing the patient to urinate freely through the expandable tubular mesh free of blockage of the urethra;
waiting a treatment time;
removing the expandable tubular mesh.

14. A method of treatment of proliferative disorders of the prostate gland comprising the steps:
placing an expandable tubular mesh into a urethra in the prostate gland to a treatment site in a urethra of a patient;
wherein the expandable tubular mesh comprises a compressed state and an expanded state;
wherein the expandable tubular mesh comprises a medicament;
wherein the expandable, tubular mesh is attached to at least a portion of a urinary catheter, wherein the urinary catheter comprises two portions: a proximal removable portion and a distal semi-permanent portion;
separating the proximal removable portion of the urinary catheter from the distal semi-permanent portion;
removing the proximal removable portion of the urinary catheter from the patient;
inserting a semi-rigid removable catheter into the patient's urethra such that a distal end of the semi-rigid removable catheter is aligned with a proximal end of the expandable tubular mesh;
pushing the semi-rigid removable catheter against the expandable tubular mesh causing the expandable tubular mesh to expand against at least a portion of the inside of the prostatic urethra;
removing the semi-rigid removable catheter from the patient;
detaching the proximal removable portion of the urinary catheter from the distal semi-permanent portion of the urinary catheter;
removing the proximal removable portion of the urinary catheter from the patient;
leaving the distal semi-permanent portion of the urinary catheter and the expanded tubular mesh in the prostatic urethra such that patent's external urinary sphincter is not blocked from voluntary control by the patient during at least a portion of treatment period;
waiting a treatment period;
wherein during at least a portion of the treatment period the patient is able to voluntarily use his external urinary sphincter to control the flow of urine from his bladder, through the expandable tubular mesh into the penile urethra;
removing the distal semi-permanent portion of the urinary catheter and the expanding tubular mesh from the patient.
